(19) United States
(12) Patent Application Publication
(10) Pub. No.: US 2002/0028237 A1
(43) Pub. Date: Mar. 7, 2002

(54) Method for reducing toxicity of a cytotoxic agent

(76) Inventors: Gail T. Colbern, Pacifica, CA (US); Karen Steinmetz, San Mateo, CA (US); Peter K. Working, Burlingame, CA (US); Alberto A. Gabizon, Jerusalem (IL)

Correspondence Address: ALZA CORPORATION, 1900 CHARLESTON ROAD BLDG M10-3, P.O. BOX 7210, MOUNTAIN VIEW, CA, 94039-7210, US

(21) Appl. No.: 09/839,918

(22) Filed: Apr. 20, 2001

Related U.S. Application Data
(60) Provisional application No. 60/199,012, filed on Apr. 20, 2000.

(51) Int. Cl. A61K 9/127; A61K 31/7068; A61K 31/704; A61K 31/513; A61K 31/48

(52) U.S. Cl. 424/450; 514/34; 514/50; 514/269; 514/288

(57) ABSTRACT

Methods for reducing the incidence and occurrence of dermal lesions in mammals, particularly human patients, who receive chemotherapy treatment and in the course of such treatment are administered liposomal formulations of cytotoxic agents are provided. Cytotoxic agents typically include doxorubicin, cytarabine, epirubicin, daunorubicin, 5-fluorouracil (5-FU) and vinorelbine. Reduction in the incidence and occurrence of dermal lesions in a patient is achieved by administration of a cytoprotective agent.

### CROSS REFERENCE TO RELATED APPLICATIONS

The complete disclosure set forth in U.S. provisional patent application entitled “A Method for Reducing Toxicity of a Cytotoxic Agent,” Ser. No. 60/199,012, filed in the United States Patent and Trademark Office on Apr. 20, 2000, is incorporated herein. The applications are commonly owned.

### BACKGROUND OF THE INVENTION

In the field of chemotherapy, a great deal of effort is currently being made to reduce and/or minimize the toxicity of cytotoxic agents administered to patients subjected to chemotherapy. In reducing the toxicity of an administered cytotoxic agent, it is believed that an improvement in the quality of life of patients subjected to chemotherapy can be increased. To achieve this end, cytoprotection of healthy tissue by, for example, thiol group donors is one of the most promising lines of research. The most extensively studied agent in the area of cytoprotection is amifostine. Amifostine is a multi-organ cytoprotector that has demonstrated cytoprotective effects both in vitro and in vivo against the most common cytotoxic drug-related toxicities and against radiation-induced adverse effects in healthy tissues.

In treatment, the difficulty and concern regarding administration of a cytotoxic agent, such as doxorubicin, is its toxicity to normal, healthy cells. Many, if not all cytotoxic agents, have the potential to mediate serious and often life-threatening toxic side effects even when given in therapeutically effective dosages. However, there are nearly always toxic side effects associated with such agents.

For example, doxorubicin is an effective cytotoxic agent developed for treating cancers, but its use may be unnecessarily limited due to toxicities. Liposomal formulations of doxorubicin, for example, Doxil® and Caelyx®, have been demonstrated to deliver doxorubicin with a lower incidence of toxicity. However, even patients receiving such liposomal formulations, conditions occur, sometimes severe in nature, such as palmar-plantar erythrodysesthesia (PPE). Presently, PPE is typically managed by dose-reduction or increased treatment interval. This approach, however, places treatment professionals in a situation that requires either discontinuation of treatment or a reduction or minimization of treatment. Clearly, a more reliable approach to reduce or minimize such conditions would be beneficial, and might permit more dose-intensive treatment strategies and avoid interruption or discontinuation of treatment.

There is, therefore, a demonstrated need for providing patients whom receive cytotoxic agents to provide a therapy aimed specifically to reduce and/or minimize the incidence and severity of resulting conditions associated with the administration of these agents. In particular, there is a need to reduce or minimize the occurrence of conditions, such as palmar-plantar erythrodysesthesia (PPE).

### DESCRIPTION OF THE FIGURES

FIG. 1 shows the scoring record for PPE lesions in rats after treatment with a liposomal formulation of doxorubicin (DOXIL®). PPE score is the product of Severity Score and percentage of body area affected. Overall PPE score is the sum of scores for each body area.

FIG. 2 shows the incidence and severity of palmar-plantar erythrodysesthesia (PPE) after treatment of rats with 2.5 mg/kg of a liposomal formulation of doxorubicin weekly for five weeks. Reduction in Overall PPE score by treatment with amifostine (110 or 200 mg/kg) concurrently with a liposomal formulation of doxorubicin. Overall PPE score is defined in FIG. 1.

FIG. 3 shows the pharmocokinetics of 10 mg/kg of a liposomal formulation of doxorubicin in female BalbC mice with or without concurrent amifostine treatment.

FIG. 4 shows the survival of female BalbC mice with IP C26 colon tumors treated once with a liposomal formulation of doxorubicin (8 mg/kg) and amifostine (200 mg/kg) before and 1, 2, 4, and 6 days after liposomal doxorubicin injection.

FIG. 5 shows the survival of female BalbC mice with IP J-6456 tumors treated once with a liposomal formulation of doxorubicin (10 mg/kg) and amifostine (50 mg/kg before and 1 and 3 days after liposomal doxorubicin injection).

FIG. 6 shows growth of SC lewis lung tumors in females B6C3-F1 mice after treatment with a liposomal formulation of doxorubicin (4 mg/kg IV, weekly for 3 cycles) and amifostine (100 or 200 mg/kg IV, before and 1, 2 and 3 days after each liposomal doxorubicin injection).

FIG. 7 shows growth of M109 footpad tumors in female BalbC mice treated with a liposomal formulation of doxorubicin (10 mg/kg) subcutaneous every other week for 2 cycles and amifostine (50 mg/kg) before and 1 and 3 days after each liposomal doxorubicin injection.

### SUMMARY OF THE INVENTION

Dermopathies, such as PPE, are a dose-limiting toxicity for liposomal preparations of cytotoxic agents, such as liposomal doxorubicin, in the treatment of solid tumors, and occurs more frequently than with i.v. bolus regimens of doxorubicin. This is possibly related to the prolonged advantageous systemic circulation of liposomal doxorubicin compared to bolus regimens of doxorubicin. The pharmacokinetics of liposomal doxorubicin mimic that of a continuous intravenous infusion of doxorubicin, a regimen under which PPE rates are also reported to be elevated (Samuels et al., Cancer Treat Rep 71:971-972 (1987); Ackland et al., Clin Pharmacol Ther 45:340-347 (1989)).

The pattern of liposomal doxorubicin related dermal toxicity in Phase II studies in solid tumors suggests that the incident and severity of liposomal doxorubicin-induced PPE is related to dose intensity and frequency. Treatment strategies that allow greater liposomal doxorubicin intensities might therefor result in increased response rates. Therefore, approaches to reduce the incident of dermopathies, such as PPE, and permit administration of liposomal doxorubicin at high dose intensities are desirable. It would also be further advantageous minimizing dermopathies without sacrificing drug efficacy.

As presented herein, treatment with cytoprotectants, such as amifostine, prior, during, or subsequent to administration of liposomal formulations of cytotoxic agents, can minimize or reduce many chemotherapy-related toxicities.

In one embodiment, a method is provided for reducing chemotherapy related dermopathies in a patient subject to treatment with a liposomal formulation containing a cytotoxic agent wherein the method includes administering to the patient an effective amount of a cytoprotective agent. Cytotoxic agent useful in the method include, for example, doxorubicin, cytarabine, epirubicin, daunorubicin, 5-fluorouracil (5-FU) and vinorelbine. Cytoprotective agents useful in the method include, for example, ergotamine, amifostine, and pyrridoxine. In one embodiment, the chemotherapy related dermopathy is plantar-planar erythrodysesthesia (PPE). Preferably, the liposomal formulation is a liposomal formulation containing polyethylene glycol (PEG) moieties.

In another embodiment, a method for reducing the occurrence of dermal lesions in a patient subject to treatment with a liposomal formulation containing a cytotoxic agent by administering to the patient an effective amount of cytoprotective agent is also provided. In yet another embodiment, a method for reducing the occurrence of plantar-planar erythrodysesthesia lesions in a patient subject to treatment with a liposomal formulation containing a cytotoxic agent by administering to the patient an effective amount of a cytoprotective agent is provided.

In another embodiment, a method for reducing the severity and incidence of a dermapathy in a patient subject to treatment with a liposomal formulation containing a cytotoxic agent by administering to the patient an effective amount of a cytoprotective agent is provided.

### DETAILED DESCRIPTION

Preparing Cytotoxic Drug Containing Liposomes

This section describes methods for preparing cytotoxic agent containing liposomes employed in the method of the invention. Cytotoxic agent containing liposomes may be prepared, for example, as described in U.S. Pat. Nos. 5,213,804 and 5,013,556, and as described herein. Specific cytotoxic agent containing liposomal formulations are commercially available. For example, DOXIL®.

Preparation of Liposomes Employed in a Method of the Invention

Liposomes used in the method of the invention are designed for use in delivering a cytotoxic agent via the bloodstream, wherein the liposomes are accessible to clearance mechanisms involving the reticuloendothelial system (RES). Sections below describe0 lipid component parameters that effect blood retention times and procedures for producing liposomes useful in the described method.

Lipid Components

The lipid components used in liposomes employed in the method of the invention may be selected from a variety of vesicle-forming lipids, typically including phospholipids and sterols. It has been demonstrated that the lipids making up the bulk of the vesicle-forming lipids in the liposomes may be either fluidic lipids, e.g., phospholipids whose acyl chains are relatively unsaturated, or more rigidifying membrane lipids, such as highly saturated phospholipids.

The vesicle-forming lipids may be selected to achieve a selected degree of fluidity or rigidity, to control the stability of the liposomes in serum and the rate of release of an entrapped cytotoxic agent from the liposomes in the bloodstream. The vesicle-forming lipids may also be selected, in lipid saturation characteristics, to achieve desired liposome preparation properties. It is generally the case, for example, that more fluidic lipids are easier to formulate and size by extrusion than more rigid lipid components, and can be readily formulated in sizes down to 0.05 microns.

Similarly, it has been found that the percentage of cholesterol in the liposomes may be varied over a wide range without significant effect on observed blood circulation lifetime. It has also been demonstrated that blood circulation lifetime is also relatively unaffected by the percentage of charged lipid components, such as phosphatidylglycerol (PG). Thus, total liposome charge may be varied to modulate liposome stability, to achieve desired interactions with or binding to particular drugs. The concentration of charged lipid may be about percent or higher.

As an example, in preparing liposomes containing an entrapped cytotoxic agent, such as doxorubicin, additional charged lipid components may be added to increase the amount of entrapped drug, in a lipid-film hydration method of forming liposomes.

The polyalkylether lipid employed in the liposomes is typically present in an amount preferably between about 1-20 mole percent, on the basis of moles of derivatized lipid as a percentage of the total moles of vesicle-forming lipids. The polyalkylether moiety of the lipid preferably has a molecular weight between about 120-20,000 daltons, and more preferably between about 1,000-5,000 daltons.

Liposomes useful in the method of the invention may be prepared by a variety of techniques, such as those described in U.S. Pat. Nos. 5,213,804 and 5,013,556. One method for preparing drug-containing liposomes is the reverse phase evaporation method described, for example, in U.S. Pat. No. 4,235,871. In this method, a solution of liposome-forming lipids is mixed with a smaller volume of an aqueous medium, and the mixture is dispersed to form a water-in-oil emulsion, preferably using pyrogen-free components. The cytotoxic agent to be delivered is typically added either to the lipid solution, in the case of a lipophilic agent, or to the aqueous medium, in the case of a water-soluble cytotoxic agent.

After removing the lipid solvent by evaporation, the resulting gel is converted to liposomes, with an encapsulation efficiency, for a water-soluble cytotoxic drug, of up to 50%. The reverse phase evaporation vesicles (REVs) have typical average sizes between about 2-4 microns and are predominantly oligolamellar, that is, contain one or a few lipid bilayer shells. The REVs may be readily sized, by extrusion to give oligolamellar vesicles having a maximum selected size preferably between about 0.05 to 0.5 microns.

To form MLV's, a mixture of liposome-forming lipids of the type detailed above dissolved in a suitable solvent is evaporated in a vessel to form a thin film, which is then covered by an aqueous medium. The lipid film hydrates to form MLVs, typically with sizes between about 0.1 to 10 microns. These vesicles, when unsized, show relatively poor blood/RES ratios, as seen in Table 9, for the unextruded MLV composition. Typically, MLVs are sized down to a desired size range of 0.5 or less, and preferably between about 0.05 and 0.2 microns by extrusion.

One effective sizing method for REVs and MLVs involves extruding an aqueous suspension of the liposomes through a polycarbonate membrane having a selected uniform pore size, typically 0.05, 0.08, 0.1, 0.2, or 0.4 microns. The pore size of the membrane corresponds roughly to the largest sizes of liposomes produced by extrusion through that membrane, particularly where the preparation is extruded two or more times through the same membrane. An additional method involves extrusion through an asymmetric ceramic filter. The method is detailed in U.S. Pat. No. 4,737,323.

Alternatively, the REV or MLV preparations can be treated to produce small unilamellar vesicles (SUVs) which are characterized by sizes in the 0.04-0.08 micron range. SUVs may be useful, for example, in targeting a tumor tissue that permits selective passage of small particles, typically than about 0.1 micron, through the capillary walls supplying the tumor. As noted above, SUVs may be formed readily from fluid vesicle-forming lipids.

After final sizing, the liposomes can be treated, if necessary, to remove free (non-entrapped) cytotoxic drug. Conventional separation techniques, such as centrifugation, diafiltration, and molecular-sieve chromatography are suitable. The composition can be sterilized by filtration through a conventional 0.45 micron depth filter.

Utility of the Method

The significantly increased circulation half-life of liposomes constructed as above can be exploited in several types of therapeutic applications. In one application, the liposome containing an entrapped cytotoxic agent is designed for sustained release of a liposome-associated agents into the bloodstream by long-life circulating liposomes. Liposomes employed in the method of the invention are typically maintained in the bloodstream up to 24 hours, and therefore sustained released of the drug at physiologically effective levels for up to about 1 day or more can be achieved. As noted above, the liposomes can be prepared from vesicle-forming lipids having a wide range of rigidifying properties, to achieve selected liposome stability and drug release rates from the liposomes in the bloodstream.

Representative cytotoxic agents useful in the method of the invention include, but are not limited to, doxorubicin, cytarabine, epirubicin, daunorubicin, 5-fluorouracil (5-FU), vinorelbine. In a preferred embodiment of the invention, the cytotoxic agent is doxorubicin or a salt thereof. And, as described below, the cytotoxic agent is associated with and/or entrapped in a liposome.

In one embodiment, liposomal doxorubicin HCL, e.g., DOXIL® and CAELYX®, (doxorubicin HCI encapsulated in long circulating, pegylated liposomes, has been successfully used for second-line therapy for patients with AIDS-associated Kaposi's sarcoma. After intravenous administration, these liposomes exhibit prolonged circulation and altered tissue distribution relative to conventional, non-pegylated liposomes (Gabizon et al., Cancer Res. 54:987-992 (1994)). Liposome encapsulation of anticancer drugs, such as the cytotoxic agents mentioned above, can enhance the therapeutic value of these cytotoxic agents, as has been demonstrated in vivo with doxorubicin HCI encapsulated in pegylated liposomes (Woodle et al., Stealth liposomes, CRC Press:Boca Raton, pg. 103-117 (1995); Vaage, Br J Cancer, 75:482-486 (1997)).

These liposomes, due to their small size, long circulation time, and reduced interaction with formed elements of the blood (Woodle et al. Biochem Biophys Acta 1113:171-199 (1992)) tend to accumulate in tumors presumably due to leakage through comprised tumor vasculature (Dvorak et al. Am J Pathol 133:95-109 (1988; Wu et al. Microvasc Res 46:231-253 (1993)).These formulations often result in higher dose administration of liposome-encapsulated without toxicity, and increased efficacy, as the encapsulated agent slowly accumulates in the tumor (Working et al. Hum Exp Toxicol 15:751-785 (1996)). In clinical trials of liposomal doxorubicin, i.e., DOXIL®, administered to patents having solid tumors, major-dose limiting toxicity occurrences include, for example, mucositis/stomatitis, and the so-called hand-foot syndrome or palmar-plantar erythrodysesthesia (PPE), as known as acral erythema. Other chemotherapy-related peripheral dermopathies have been reported under several synonyms: Burgdorf's syndrome, chemotherapy-induced acral erythema, hand-foot syndrome, PPE and toxic erythema of the palms and soles. Cytarabine, doxorubicin HCI (particularly as a long-term intravenous infusion), and 5-fluorouracil (5-FU) are the most common cytotoxic agents associated with these dermopathies, but the reaction has also been reported with various other agents and is most often associated with protracted infusions of the chemotherapeutic agent (Lokich et al. Ann Intern Med 101:798-800 (1984); Vogelzang Ann Intern Med 103:303-304 (1985)).

For example, the incident of PPE observed in clinical studies of 5-FU-containing regimens varies from approximately 7% to over 50%, depending on regimen and dosage, but no clear relationship to dose intensity has been demonstrated (Chiara et al. Eur J Cancer 33:967-969 (1997); Tralongo et al. Anticancer Res 15:635-638 (1995)). A similar incidence of Grade 3 PPE has been reported in a study of Xeloda®, a 5-FU precursor currently awaiting approval by the FDA.

PPE typically begins with tingling hands and feet and progresses over 3 to 4 days to discomfort and pain, with swollen and erythematous palms and soles and tenderness, particularly in the distal phalanges. The macular reddening primarily involves the palms and soles, but may also include the sides and dorsal surfaces of the hands and feet and other sites, and may be associated with pain and blistering. The healing process usually includes superficial desquamation of the involved skin and reepithelialization (Jucgla et al. J Clin Oncol 15:3164 (1997))

PPE has been observed and describe in patients who received intensive chemotherapy with single agents or combination regimens. This is illustrated, for example, in case reports wherein the following cytotoxic agents were used: 5-FU i.v. bolus given to a patient with metastatic colon adenocarcinoma (lurlo Acta Oncologica 36:653-654 (1997); hydroxyurea given to one patient for chronic myelogenous leukemia Silver et al. 98:675 (1983)); etretinate therapy in 5 psoriatic patients (David et al. Acta Derm Venereol (Stockh) 66:87-89 (1986)) cytarabine in a case of acute myelogenous leukemia (Rongioletti et al. J Cutan Pathol 18:453-456 (1991)) etoposide-containing regimens in a case of small-cell lung cancer (Portal et al. Cancer Chemother Pharmacol 34:181 (1994)) Taxol in a case of stage IIA breast carcinoma (De Argila et al. Dermatology 192:377-378 (1996); Tegafur (fluorinated pyrimidine analogous to 5-FU) in two cases of g.i. adenocarcinoma (Rios-Buceta et al. Acta Derm Venereol (Stockh) 77:80-81 (1997)); Taxotere, in several cases (Vukelja et al. J Natl Cancer Inst 85:1432-1433 (1993); (Zimmerman et al. South Med J 86(9 Suppl 1):S20-S21 (1993))

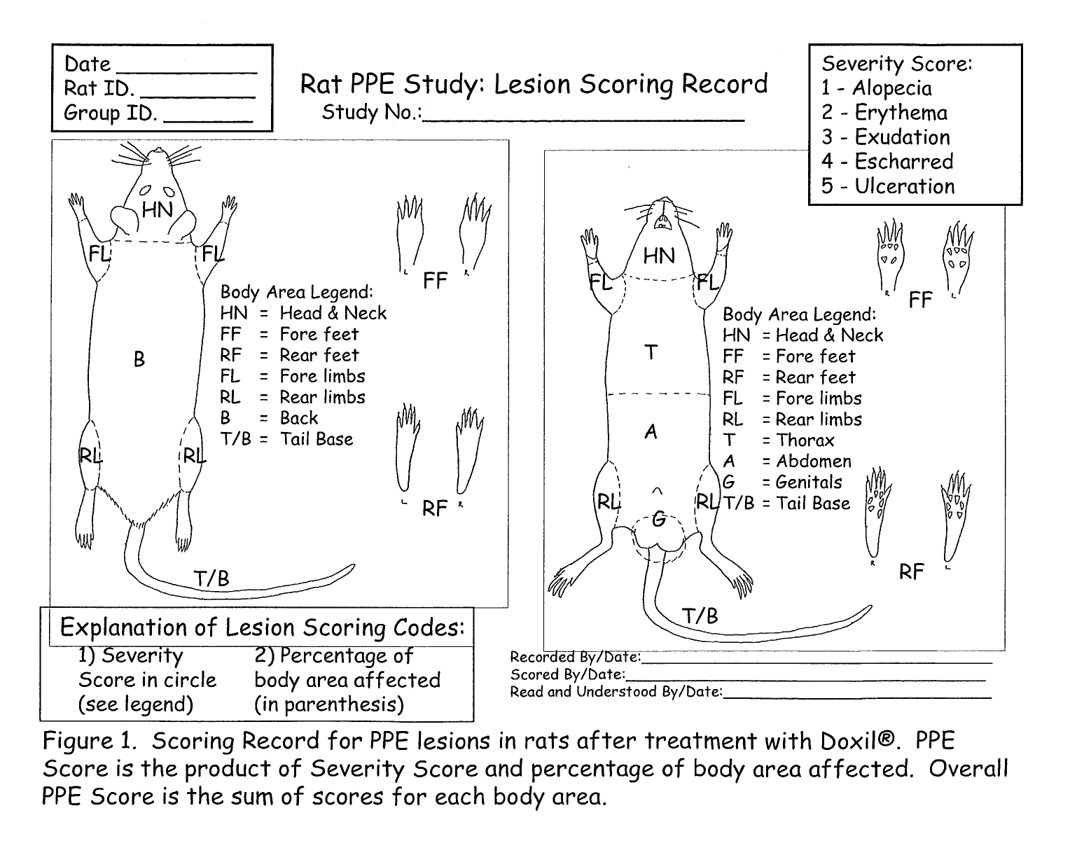

Pharmacokinetic parameters of liposomal doxorubicin in mice were not significantly affected by concurrent treatment with amifostine. For example, rats given 2.5 mg/kg liposomal doxorubicin weekly for five weeks develop PPE histopathologically similar to PPE in humans. Lesions of PPE are typically scored by size and severity and presented as a total PPE score for each animal (FIG. 1). Treatment with amifostine (200 mg/kg IV) prior to liposomal doxorubicin or amifostine (100 mg/kg IV) prior to and four days after liposomal doxorubicin significantly reduced the severity score (from 12.99 for liposomal doxorubicin to 3.18 and 4.33 for the two amifostine treatment regimens, respectively; p<0.02). Incidence of PPE was also reduced.

Antitumor activity of liposomal doxorubicin with concurrent amifostine treatment was investigated in four mouse tumor models: C26 colon, Lewis lung, M109 lung and J-6456 lymphoma (Examples 4-7). Although mice do not develop PPE and, thus, the effect of amifostine on PPE could not be evaluated, equivalent allometric dosing was used in these studies. Liposomal doxorubicin had significant antitumor activity, determined as survival (C26 and J6456 tumors), or reduction in rate of tumor growth (Lewis lung and M109 tumors), compared with saline-treated controls in all studies. Simultaneous treatment with liposomal doxorubicin (4 to 10 mg/kg IV for one to three cycles) and amifostine (50 to 200 mg/kg IV for one to four treatments per cycle) did not decrease the antitumor activity of Doxil in any of the studies. Thus, in preclinical models, amifostine is effective in reducing severity and incidence of PPE induced by liposomal preparations of a cytotoxic agent such as doxorubicin, and does not alter its antitumor efficacy or pharmacokinetics.

PPE

Palmar-plantar erythrodysesthesia or hand-foot syndrome is frequently observed in patients treated with continuous infusion of cytotoxic agents such as 5-fluorouracil, vinorelbine, or doxorubicin. Initial signs of PPE include tingling in the extremities, followed by redness, edema, exfoliation, and scaling that can eventually lead to inflammation, dermatitis, and skin ulceration. PPE lesions differ from the severe tissue necrosis from inadvertent extravasation of a chemotherapeutic agent in that the latter occurs locally adjacent to the point-of-entry and is associated with direct contact of the cytotoxic agent with the tissue. PPE is also a major dose-limiting toxicity (DLT) in liposomal cytotic agents administered to cancer patients. The current clinical strategy is either to remove a patient completely from liposomal doxorubicin treatment or reduce the dose. Either of these alternatives means that the tumors potentially are not being treated as aggressively as possible. The pathogenesis of PPE is poorly understood, and very few effective treatments are available to patients with these lesions.

DLTs of cancer chemotherapy lead to both reductions in quality of life for patients and limited antitumor response due to reduction in chemotherapy dose intensity. Cytoprotectants have been developed to reduce toxicities and allow continued dose-intensive therapy. Cytotoxic agents useful in the present invnetion include, for example, ergotamine, pyrridoxine, and amifostine. Amifostine (Ethyol®) is a prodrug that requires activation by dephosphorylation to produce the free thiol, WR-1065, and other active metabolites. The enzyme required for this conversion, capillary alkaline phosphatase, is preferentially present in normal tissue at higher concentrations than in tumors. Conversion produces a thiol group donor that provides an alternative target for alkylating agents, as well as acting as a scavenger of oxygen free radicals. Thus, amifostine provides cytoprotection preferentially to normal tissues and cells during both radiation and chemotherapy. If amifostine allows maintenance of dose-intensive chemotherapy with liposomal cytotoxic agent formulations substantial improvement may be achieved in clinical outcome, as shown herein, and specifically in the Examples. A model for liposomal doxorubicin induced PPE has been developed in adult male rats. In this model, the incidence and progression of PPE after Doxil® treatment is very similar to that observed in human patients.

Experiments described herein were designed to demonstrate the efficacy of amifostine in reducing PPE lesions induced by liposomal cytotoxic agents in the rat model. Subsequently, studies were performed to demonstrate the effect of simultaneous administration of liposomal doxorubicin and amifostine on the pharmacokinetics and antitumor efficacy of liposomal doxorubicin in murine tumor models.

Liposomal Doxorubicin Preclinical Studies

Administration of liposomal doxorubicin is associated with the appearance of PPE-like dermal lesions, primarily on the feet and legs, of rats, rabbits and dogs that receive multiple treatments. Similar degenerative changes in the skin have also been described in hamsters given intraperiotenial injections of non-liposomal doxorubicin HCI three times weekly for up to 4 weeks (Dantchev et al. Cancer Treat Rep 63:875-888(1979)). No evidence of dermal lesions has been observed in animals treated with placebo liposomes. Thus, the cutaneous lesion in liposomal doxorubicin treated animals is consistent with a known dermatologic toxicity of doxorubicin HCI. PPE is believed to represent the response of the dermis to long-term exposure to low levels of doxorubicin, as occurs during continuous infusion of adriamycin or administration of liposomal doxorubicin.

The animal studies described in the Examples, have shown that PPE is readily reversible upon cessation of treatment, and that its severity is significantly lessened by lengthening the dosing interval (e.g., from 1 week to 3 weeks). Microscopic examination of the skin lesions reveals histological features of focal parakeratosis, acantholysis, and chronic active inflammation affecting the skin and underlying dermis. The affected sites are histologically unremarkable by study termination in the recovery animals, even in the absence of therapy.

According to a pharmacodynamic model developed in a study of dogs treated with liposomal doxorubicin at various doses and time intervals, minimizing the dose intensity within a treatment regimen is the key factor in decreasing the probability of dermal lesion development (Newman et al. Manuscript submitted for publication, 1998). The onset of lesions typically occur within 1 to 2 weeks after treatment is begun, and lesions start to heal at rates that vary depending both on lesion severity and dose frequency. Higher dose intensities (high does levels or low dose levels given frequently) cause more sever lesions, whereas increased interval between dosing minimizes the incidence and severity of the dermal lesions.

Anecdotal findings for patients receiving continuous infusions of 5-FU, for example, have suggested that oral pyridoxine in daily does of 50 to 150 mg may palliate PPE (Vukelja et al. Ann Intern Med 111:688-689 (1989); Fabian et al. Invest New Drugs 8:57-63 (1990)). A recent study in dogs with canine non-Hodgkin's lymphoma evaluated the potential of pyridoxine to ameliorate PPE in a double blind study (Vail et al. Clin Cancer Res, submitted). Dogs receiving 1 mg/kg liposomal doxorubicin (q 3 weeks for 5 treatments), were randomized to receive daily oral pyridoxine (50 mg tid for 15 weeks) or placebo capsules of identical size and color. No difference was observed in remission rates between groups, but the likelihood of developing severe PPE and having to discontinue liposomal doxorubicin treatment was 4.2 fold (relative risk) more likely in placebo-treated dogs than in dogs that received pyridoxine (p=0.032). A trend to longer-lasting remission was seen in pyridoxine-treated dogs (159 days, pyridoxine; 48 days, placebo; p=0.084). Similarly, survival showed a trend to be extended by pyridoxine treatment (201 days, pyridoxine; 130 days placebo; p=0.182). These trends are probably due to the higher cumulative doses of liposomal doxorubicin that could administered to pyridoxine treated dogs (median: 4.7 mg/kg) than to the placebo-treated dogs (median: 2.75 mg/kg). Studies in several murine and human xenograft tumor models have demonstrated that pyridoxine co-treatment does not reduce the therapeutic effect of liposomal doxorubicin (Colbern et al. Proc ASCO 1998, submitted)

Liposomal Doxorubicin Clinical Experience

PPE in AIDS-related Kasposi's Sarcoma Patients

PPE has been observed in liposomal doxorubicin treated patients (Gordon et al. Cancer 75:2169-2173 (1995); and is not frequent for patients treated with i.v. bolus regimens of doxorubicin. A small percentage, 3.4% (24/705), of patients with AIDS-related Kaposi's sarcoma (KS) developed PPE upon treatment with liposomal doxorubicin at a dose of 20 mg/m² every 2 to 3 weeks (DOXIL Package Insert. Aug. 4, 1997). Experience to date has shown that in most patients, the reaction is mild and occurs after 6 or more weeks of treatment and resolves in 1 to 2 weeks with interruption or discontinuation of therapy. In some patients, however, PPE can be severe and debilitating. Data from studies of liposomal doxorubicin administered to patients with AIDS-KS revealed that 3 of 705 patients (0.4%) discontinued therapy because they developed clinical hand-foot syndrome. The incident of hand foot syndrome may be higher when liposomal doxorubicin is administered at higher doses or at shorter intervals.

PPE in Ovarian Cancer patients

In a Phase II study of liposomal doxorubicin administered at doses of 50 mg/m² every 3 weeks to patients with ovarian cancer who failed to respond to regimens based on platinum and paclitaxel, grade 3 PPE was observed in 1/35 (29%) patients (Muggia et al. J Clin Oncol 15:987-993 (1997)) All of these patients eventually required dose reductions and dose delays, which were successful in alleviating the signs and symptoms of PPE. The liposomal doxorubicin regimen was modified to 40 m/mg² every 3 or 4 weeks, occasionally even 5 weeks, and the skin toxicities resolved by 5 weeks after dosing. Prophylactic measures such as avoidance of tight shoes and exercise during the first week after dosing and other symptomatic measures appeared to be of uncertain benefit. This phase II study in patients with refractory ovarian cancer showed a response rate of 26% (9/35), with one complete and eight partial responses.

Additionally, PPE developed in a patient with advanced carcinoma of the ovary despite i.v. premedication with dexamethasone, diphenhydramine, and cimetidine prior to liposomal doxorubicin 50 mg/m² q 4 weeks. In this case, premedication with oral dexamethasone 8 mg bid prevented further episodes of PPE and allowed continuation of treatment (Titgan MA Proc ASCO 16:82a Abstract 288 (1997))

PPE in Breast Cancer Patients

In a phase II study of liposomal doxorubicin administered at doses of 45 to 60 mg/m² every 3 to 4 weeks to patient with stage IV breast cancer, skin toxicity of grade 3 or higher appeared to be more frequent in patients receiving liposomal doxorubicin at higher dose intensities (Ranson et al. J Clin Oncol 15:3185-3191 (1997)). Thus, such skin toxicities occurred in 7/13 (54%) patients dosed at 60 mg/m² every 3 weeks, in 12/26 (46%) patients dosed at 45 mg/m² every 3 weeks, and in 5/32 (16%) patients dosed at 45 mg/m² every 4 weeks. In all cases, the skin toxicity was found to be reversible. This phase II study in patients with advanced breast cancer showed a response rate of 31% (20/64), with 4 complete and 16 partial responses.

Strategy for Managing PPE during Liposomal Doxorubicin Therapy

For AIDS-KS patients, the incidence of PPE may be higher when liposomal doxorubicin is administered at doses that are higher or at intervals that are shorted than those recommended.

For solid tumor patients enrolled in clinical trials of liposomal doxorubicin given at higher does than those used for AIDS-KS patients, dose modifications may also be appropriate when patients develop PPE. Such dose modifications are summarized below for PPE occurring 4, 5, or 6 weeks after dosing. The following points should be noted in connection with a 4-week treatment cycle.

(1) patients with grade 1 PPE, without previous grade 3 or 4 skin toxicity, may continue treatment at the same dose at 4 week intervals; (2) for patients who had grade 3 or 4 toxicity that has resolved to grade 1, the dose should be delayed for a week; (3) if grade 1 toxicity persists after the dose has been delayed for 2 weeks (6 weeks since the last dose), the dose would be reduced by 25% and dosing be continued at 4-week intervals; (4) if grade 2 or greater PPE occurs on the scheduled day for dosing, the dose should be delayed for 1 week; (5) if grade 2 or greater PPE persists after the dose has been delayed for 1 week, dosing should be delayed for an additional week; (5) if grade 2 toxicity persists after the dose has been delayed for 2 weeks (6 weeks since the last does), then treatment should be resumed at 4 week intervals with liposomal doxorubicin at a 25% reduced dose; (5) if grade 3 or 4 toxicity persists for 2 weeks beyond the next scheduled dose (6 weeks since the last dose), study drug therapy should be discontinued.

The following examples illustrate methods of the invention. These examples are intended to illustrate specific methods of the invention, but are in no way intended to limit the scope thereof.

### EXAMPLES

### Example 1

Toxicology of Rat Model of PPE

24 male Sprague-Dawley rats weighing between 250 to 300 grams(g) were intravenously (IV) treated with Doxil® weekly for 5 cycles of administration. The dosage administered was 2.5 mg/kg IV. All 24 rats developed PPE. The rats were observed weekly for lesions and scoring (FIG. 1). Scores for severity and percentage affected body area were analyzed and reported on final observation.

### Example 2

Chemoprotection—Administration of the Cytoprotectant Amifostine

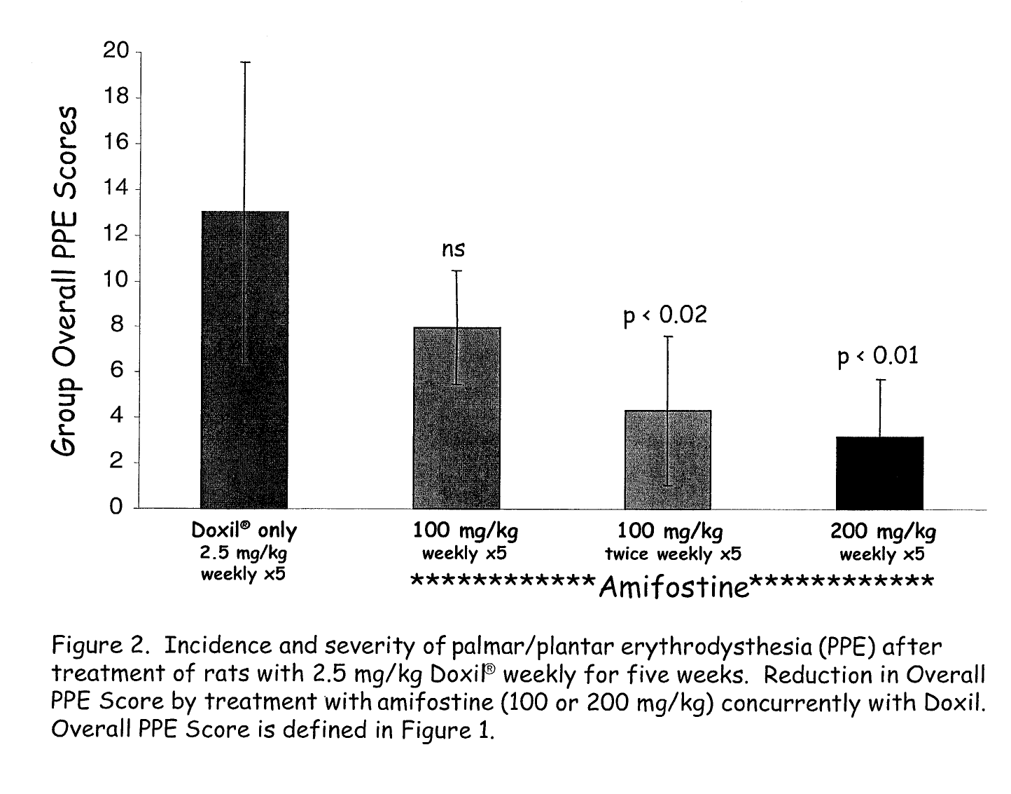

Sprague-Dawley rats were treated with amifostine at a dosage of 100 or 200 milligram/kilogram (mg/kg) IV, weekly, approximately 15 minutes before each Doxil® injection or treated with amifostine, 100 mg/kg IV, twice weekly at 15 min before and 3 days after Doxil injection (FIG. 2).

Rats treated with Doxil® consistently developed skin lesions (overall severity score of 12.99). Both gross and histopathologic appearance were similar to human PPE (data not shown). Treatment with amifostine prior to Doxil® reduced overall severity score (7.94 (not significant), 4.33 (p<0.02), and 3.18 (p<0.01) for amifostine 100 mg/kg weekly, 100 mg/kg twice weekly and 200 mg/kg weekly, respectively). Response was dose dependent, with 200 mg/kg amifostine providing the best effect.

### Example 3

Pharmacokinetics

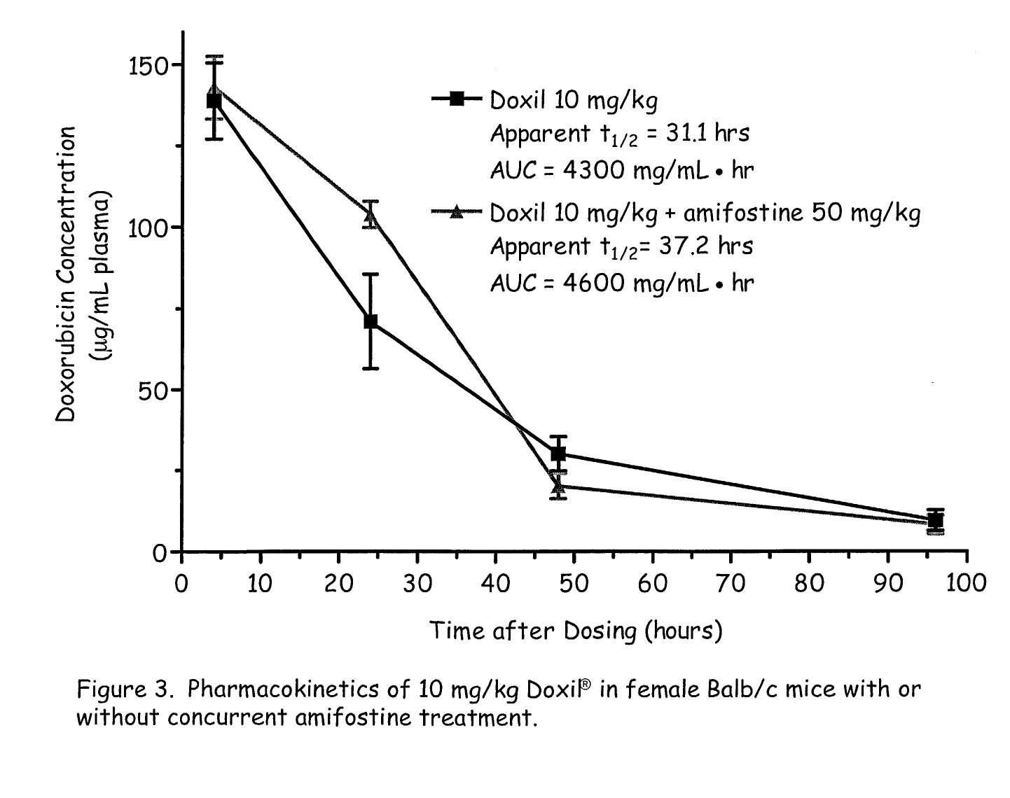

30 BalbC, female mice approximately 8-9 weeks old received a single dose of Doxil®, 10 mg/kg IV, and a single dose of amifostine, 50 mg/kg at 30 minutes before DOXIL® administration and at 24 and 72 hours after Doxil® administration (FIG. 3).

b. Plasma was collected from each of the 30 BalbC mice at 4 hours, 24 hours, 48 hours, and 96 hours after Doxil® injection. Analysis for doxorubicin.

c. Plasma samples were collected (200 milliliters (ml)), and was subsequently diluted 1:10 in HCI acidified isopropanol and incubated overnight at 4° C. Samples were subsequently centrifuged and the supernatants were collected for direct fluorimetry (Ex 470 nm-Em 590 nm).

d. Plasma pharmacokinetics after treatment with 10 mg/kg Doxil® were similar to those previously reported (FIG. 3). Apparent t_{½}=31.1 hours, AUC=4300 mg/mL·h. Plasma pharmacokinetics after treatment with 50 mg/kg amifostine followed by 10 mg/kg Doxil® were equivalent. Apparent t_{½}=37.2 hours, AUC=4600 mg/mL·h

### Example 4

Models with Survival Endpoint

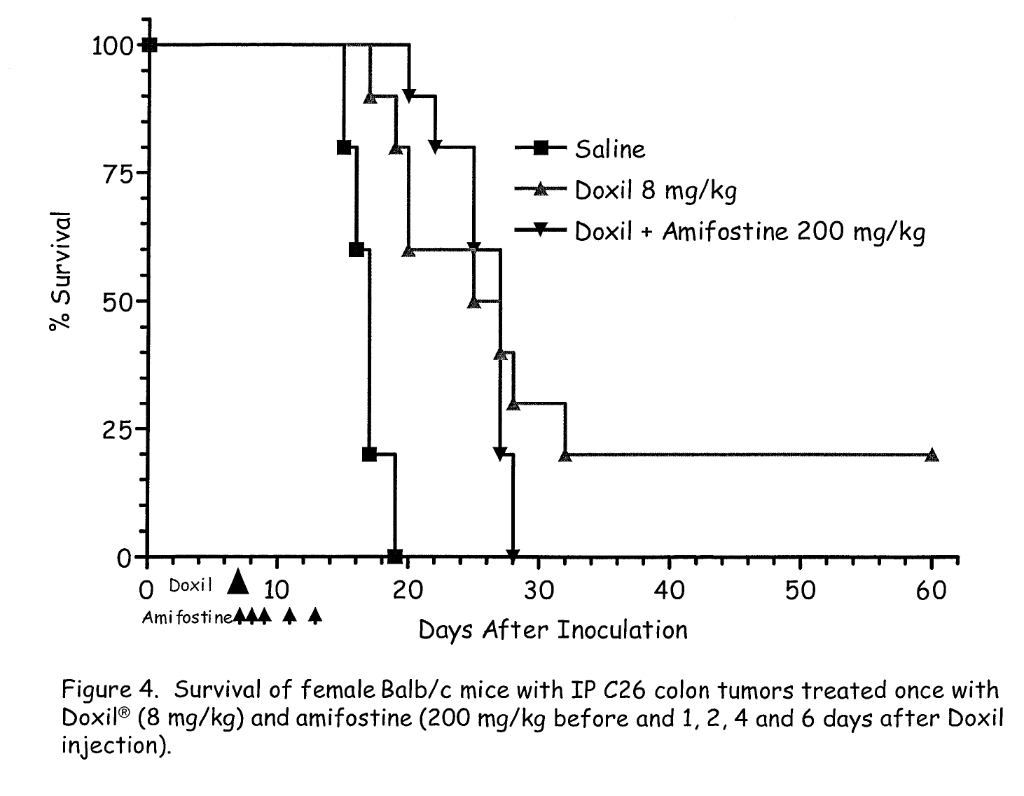

Euthanasia was provided for animals with weight loss greater than 20% or with clinical signs of distress. Survival of female BalbC mice with IP C26 colon tumors treated once with Doxil® (8 mg/kg) (tumor inoculation: 10⁶ cells IP). Animals were administered 200 mg/kg of amifostine subcutaneously (SC) prior to Doxil® administration and 1, 2, 4 and 6 days after Doxil® administration (FIG. 4).

In the C26 colon model (FIG. 4), prolongation of survival similar for Doxil® (26 days) and Doxil+Amifostine (27 days) (p=0.476). Treated mice survived longer than controls (17 days). Significant toxicity (weight loss, lethargy) with amifostine at 200 mg/kg for five administrations, dose was reduced in subsequent studies.

### Example 5

J-6456 lymphoma tumors in BalbC female mice

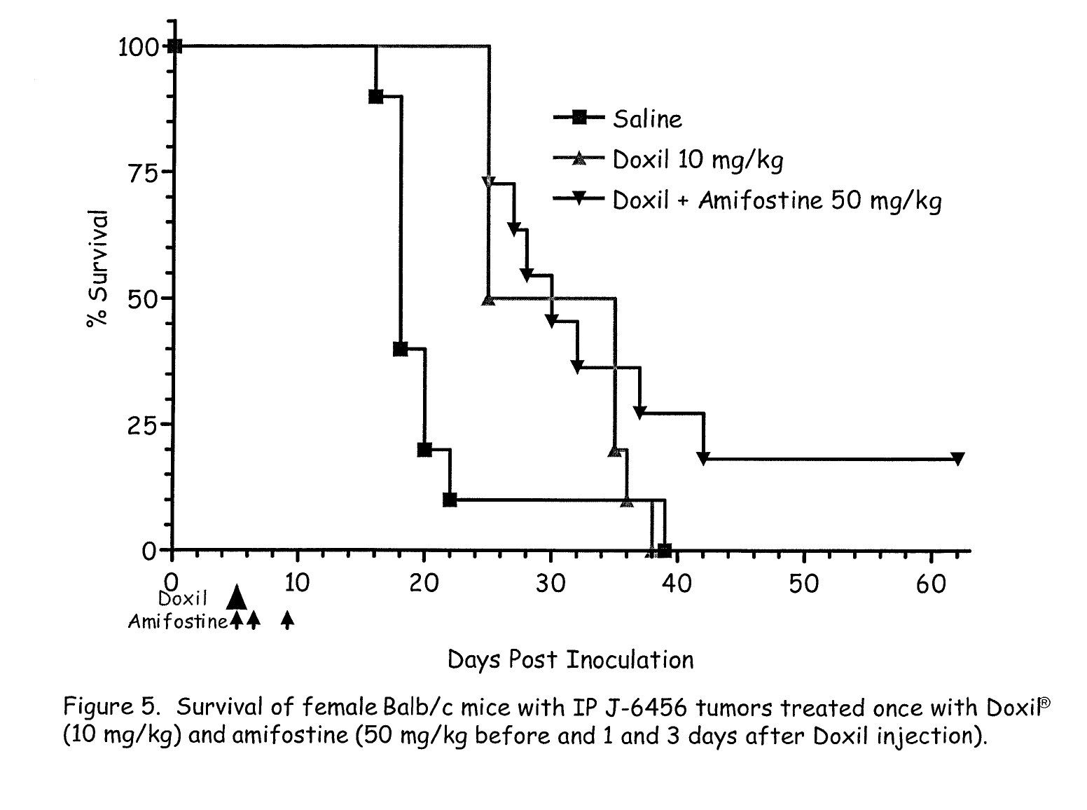

10⁶ cells injected IP. Doxil® was administered in a single does, 10 mg/kg IV, on the fifth day after tumor inoculation. Amifostine was administered at a dosage of 200 mg/kg SC, before and 1, and 3 days after Doxil® injection (FIG. 5).

Prolongation of survival similar for Doxil® (30 days) and for Doxil®+Amifostine (30 days) (p=0.282). Treated mice survived longer than controls (18 days). Two of the Doxil®+Amifostine-treated mice were tumor free at 65 days.

### Example 6

Models with tumor growth endpoint

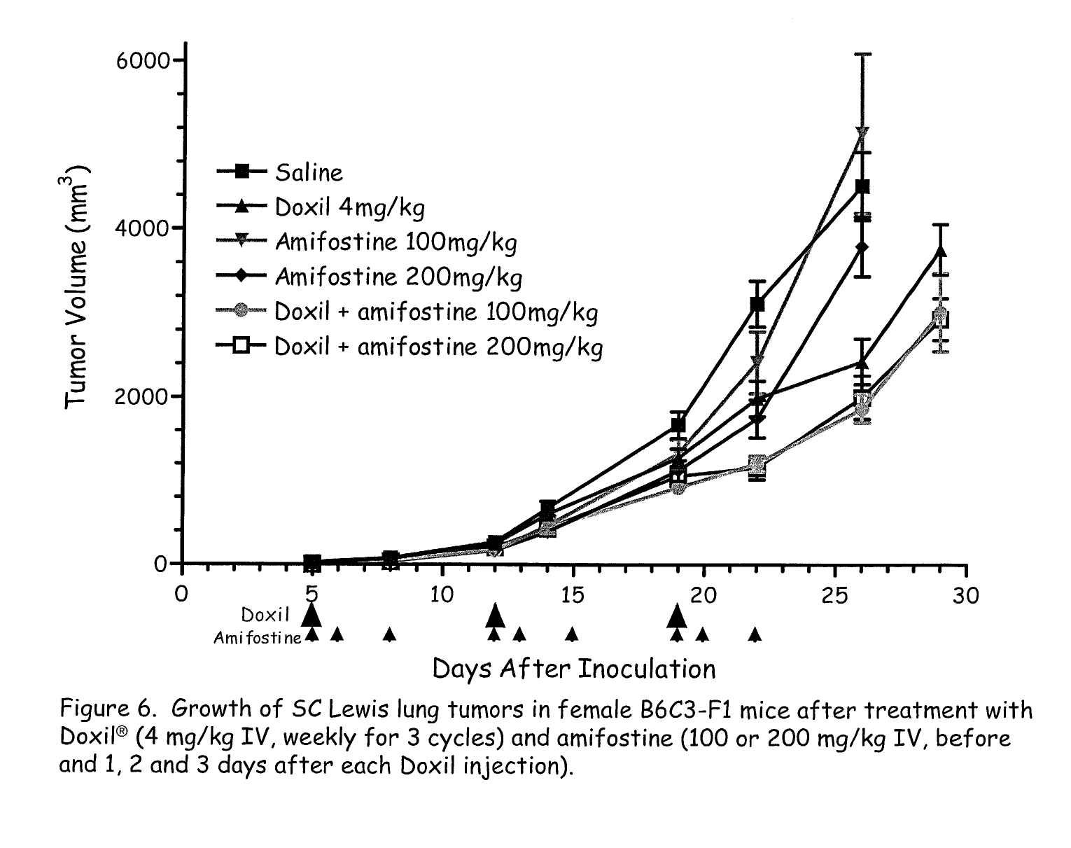

10⁶ cells were injected for tumor inoculation (SC Lewis lung tumors in B6C3-F1 female mice) (FIG. 6). Doxil® was administered 4 mg/kg IV, weekly for 3 cycles. Amifostine, 100 or 200 mg/kg IV, was administered before and 1, 2, and 3 days after each Doxil® treatment cycle. Euthanasia was provided for animals demonstration weight loss greater than 20%, tumor volume greater than 4,000 mm³ or clinical signs of distress.

Lewis lung model (FIG. 6).

Doxil® significantly reduced rate of tumor growth (p=0.0002). Amifostine alone did not affect rate of tumor growth (p=0.437). Doxil+Amifostine reduced rate of tumor growth significantly more than Doxil alone (p=0.0003). Apparent amifostine enhancement of Doxil® antitumor efficacy was not dose responsive (p=0.658). Amifostine (200 mg/kg days 0-3 of each Doxil treatment cycle) was not significantly toxic to the animals.

### Example 7

Madison 109 (M109) lung tumors in BalbC female mice

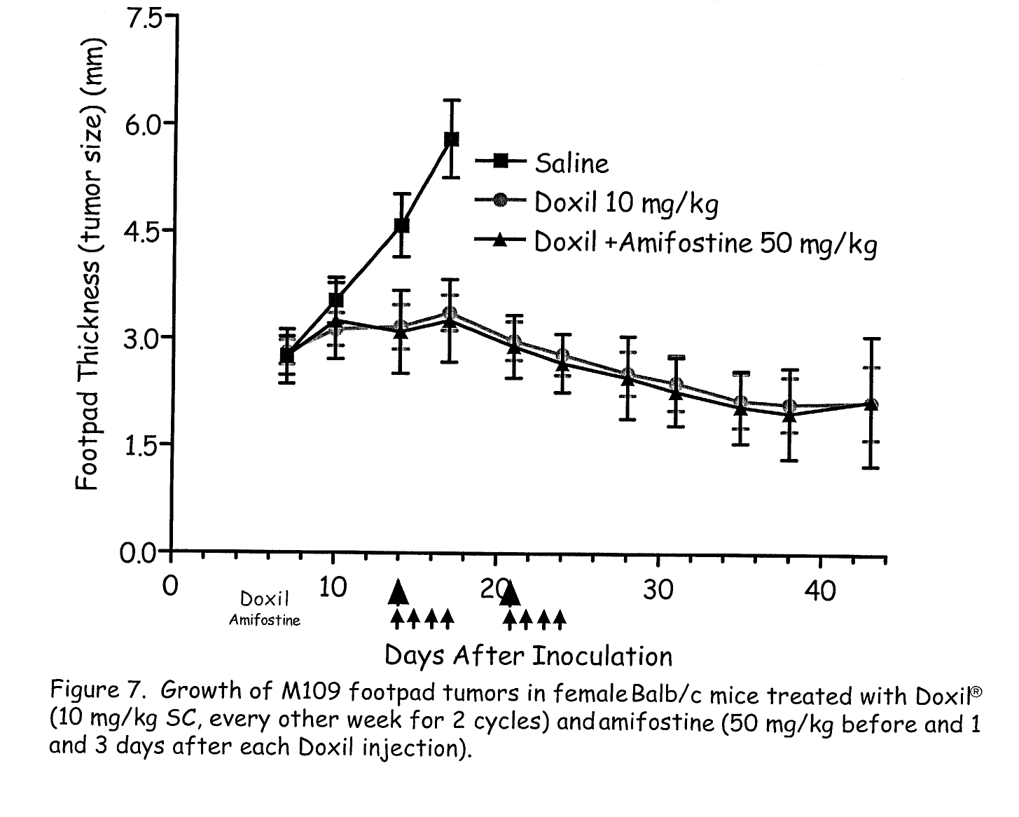

10⁶ cells were injected for tumor inoculation in right front footpad. Tumor growth was assessed as footpad thickness. Doxil® was administered, 10 mg/kg IV, every other week for 2 cycles. Amifostine was administered, 50 mg/kg SC, before and 1 and 3 days after each Doxil® treatment cycle (FIG. 7).

Doxil® significantly reduced rate of tumor growth (p<0.0001). Doxil+Amifostine reduced rate of tumor growth similar to Doxil® alone (p=0.793).

The complete disclosures of the patents, patent documents, publications cited herein are incorporated by reference in their entirety as if each were individually incorporated. Various modifications and alterations to this invention will become apparent to those skilled in the art without departing from the scope and spirit of this inventions. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method for reducing chemotherapy related dermopathies in a patient subject to treatment with a liposomal formulation comprising a cytotoxic agent comprising administerting to the patient an effective amount of a cytoprotective agent.

2. The method of claim 1 wherein the cytotoxic agent is selected from the group consisting of doxorubicin, cytarabine, epirubicin, daunorubicin, 5-fluorouracil (5-FU) and vinorelbine.

3. The method of claim 2 wherein the cytotoxic agent is doxorubicin.

4. The method of claim 1 wherein the cytoprotective agent is selected from the group consisting of ergotamine, amifostine, and pyrridoxine.

5. The method of claim 4 wherein the cytoprotective agent is amifostine.

6. The method of claim 1 wherein the chemotherapy related dermopathy is plantar-planar erythrodysesthesia (PPE).

7. The method of claim 1 wherein the liposomal formulation is a liposomal formulation comprising polyethylene glycol (PEG) moieties.

8. A method for reducing the occurrence of dermal lesions in a patient subject to treatment with a liposomal formulation comprising a cytotoxic agent comprising administering to the patient an effective amount of cytoprotective agent.

9. The method of claim 8 wherein the cytotoxic agent is selected from the group consisting of doxorubicin, cytarabine, epirubicin, daunorubicin, 5-fluorouracil (5-FU) and vinorelbine.

10. The method of claim 9 wherein the cytotoxic agent is doxorubicin.

11. The method of claim 8 wherein the cytoprotective agent is selected from the group consisting of ergotamine, amifostine, and pyrridoxine.

12. The method of claim 11 wherein the cytoprotective agent is amifostine.

13. The method of claim 8 wherein the chemotherapy related dermopathy is plantar-planar erythrodysesthesia (PPE).

14. The method of claim 1 wherein the liposomal formulation is a liposomal formulation comprising polyethylene glycol (PEG) moieties.

15. A method for reducing the occurrence of plantar-planar erythrodysesthesia lesions in a pateint subject to treatment with a liposomal formulation comprising a cytotoxic agent comprising administering to the patient an effective amount of a cytoprotective agent.

16. The method of claim 15 wherein the cytotoxic agent is selected from the group consisting of doxorubicin, cytarabine, epirubicin, daunorubicin, 5-fluorouracil (5-FU) and vinorelbine.

17. The method of claim 16 wherein the cytotoxic agent is doxorubicin.

18. The method of claim 15 wherein the cytoprotective agent is selected from the group consisting of ergotamine, amifostine, and pyrridoxine.

19. The method of claim 18 wherein the cytoprotective agent is amifostine.

20. The method of claim 15 wherein the liposomal formulation is a liposomal formulation comprising polyethylene glycol (PEG) moieties.

21. A method for reducing the severity and incidence of a dermapathy in a patient subject to treatment with a liposomal formulation comprising a cytotoxic agent comprising administering to the patient an effective amount of a cytoprotective agent.

22. The method of claim 21 wherein the cytotoxic agent is selected from the group consisting of doxorubicin, cytarabine, epirubicin, daunorubicin, 5-fluorouracil (5-FU) and vinorelbine.

23. The method of claim 22 wherein the cytotoxic agent is doxorubicin.

24. The method of claim 21 wherein the cytoprotective agent is selected from the group consisting of ergotamine, amifostine, and pyrridoxine.

25. The method of claim 24 wherein the cytoprotective agent is amifostine.

26. The method of claim 21 wherein the chemotherapy related dermopathy is plantar-planar erythrodysesthesia (PPE).

27. The method of claim 1 wherein the liposomal formulation is a liposomal formulation comprising polyethylene glycol (PEG) moieties.

28. A method for reducing chemotherapy related dermopathies in a patient subject to treatment with a liposomal formulation comprising doxorubicin comprising administering to the patient an effective amount of amifostine.

29. A method for reducing the occurrence of dermal lesions in a patient subject to treatment with a liposomal formulation comprising doxorubicin comprising administering an effective amount of cytoprotective agent.

30. A method for reducing plantar-planar erythrodysesthesia lesions in a patient subject to treatment with a liposomal formulation of doxorubicin comprising administering an effective amount of amifostine.

31. A method for reducing the severity and incidence of a dermapathy in a patient subject to treatment with a liposomal formulation of doxorubicin comprising administering to the patient an effective amount of a cytoprotective agent
